(19) United States
(12) Patent Application Publication
(10) Pub. No.: US 2023/0414821 A1
(43) Pub. Date: Dec. 28, 2023

(54) DEVICE AND METHOD FOR ATTENUATING AND/OR KILLING MICROORGANISMS, VIRUSES, VIRIONS, PRIONS, ALLERGENS AND PSEUDOALLERGENS AND/OR FOR BLOCKING THEIR TRANSMISSION PATHS

(71) Applicant: Gregor Luthe, Costa da Caparica (PT)

(72) Inventor: Gregor Luthe, Costa da Caparica (PT)

(21) Appl. No.: 18/033,476

(22) PCT Filed: Oct. 21, 2021
(86) PCT No.: PCT/EP2021/025419

§ 371 Date: Apr. 24, 2023

(30) Foreign Application Priority Data
Oct. 24, 2020 (DE) 10 2020 006 520.1

(51) Int. Cl. A61L 9/20; A61L 9/014; F24F 8/22

(52) U.S. Cl. CPC A61L 9/20; A61L 9/014; F24F 8/22; A61L 2209/12; A61L 2209/11; A61L 2209/14; A61L 2209/16

(57) ABSTRACT

The invention relates to a device 1 shielded against emissions of actinic radiation according to FIG. 2 for attenuating and/or killing and/or chemically and/or physicochemically modifying microorganisms, viruses, virions, prions, allergens and pseudoallergens and/or their residues and/or decomposition products and/or for blocking their transmission pathways by means of actinic radiation and subsequent acoustophoretic treatment, comprising (i) an intake region 2 having an intake opening 2.3 for contaminated air 2.2, (ii) an air conveying region 3 having an axial rotor V or a fan, (iii) an irradiation region 4 having a radiation source 4.1, (iv) a power supply region 5 having a holder 5.2 with power lines for the power supply 5.1 of the radiation source 4.1, and (v) openings 6.5 for the entry of the irradiated air 6.5.1 into an acoustophoresis region 6 with an acoustophoresis device 6.6 for generating a stationary acoustic ultrasonic field, wherein the acoustophoresis device 6.6 represents a wall-free flow region 6.6.1 or a flow tube 6.6.2 with a closed wall 6.6.3 enclosing a flow channel 6.6.4, (vi) electronics E for generating, monitoring and stabilizing a feedback loop for adjusting and stabilizing the stationary acoustic ultrasonic field, (vii) an air outlet region 7 shielding actinic radiation, and (viii) an air outlet region 8 for the treated air 8.2 containing the attenuated and/or killed and/or chemically and/or physicochemically modified microorganisms, viruses, virions, prions, allergens and pseudoallergens and/or their residues and/or decomposition products 8.2.1; method for attenuating and/or killing and/or chemically and/or physically chemically modifying microorganisms, virions, virions, prions, allergens and pseudoallergens and/or their residues and/or decomposition products 8.2.1 as well as the use of the device 1 and the method.

The present invention relates to a device for attenuating and/or killing microorganisms, in particular pathogenic microorganisms, viruses, virions, prions, allergens and pseudoallergens and/or for blocking their transmission pathways.

Furthermore, the present invention relates to a method for attenuating and/or killing microorganisms, viruses and virions, in particular pathogenic microorganisms, viruses, virions, prions, allergens and pseudoallergens and/or for blocking their transmission pathways.

Furthermore, the present invention relates to the use of the device and method for the production of room air containing attenuated and/or killed microorganisms and viruses, in particular attenuated and/or killed pathogenic microorganisms, viruses, virions, prions, allergens and pseudoallergens, and/or wherein their transmission pathways are blocked, Description of the prior art Microorganisms are archaea, bacteria, eukaryotes, protists, fungi and green algae. It is still a subject of debate whether viruses or virions should be considered organisms at all, Pathogenic microorganisms and viruses are the source of a number of serious diseases, epidemics and pandemics such as the current Covid-19 pandemic.

Numerous pesticides such as fungicides, herbicides, insecticides algaecides, molluscicides, rodenticides, acaricides and slimicides have been developed to combat the harmful effects on multicellular human animals and plants.

Similarly, numerous antimicrobial agents such as germicides, antibiotics, bactericides, virucides, antifungals, antiprotozoal agents and antiparasitic agents have been developed to cure the diseases caused by the microorganisms.

The permanent threat of microorganisms, especially viruses and virions, and more specifically the SARS-Co-V2 coronavirus, has created a growing demand for efficient and effective methods of decontamination and disinfection. The “List N: Products with Emerging Viral Pathogens AND Human Coronavirus Claims for Use against SARS-CoV-2, Date Accessed: May 31, 2020 of the EPA lists, US Govt.,” lists numerous organic and inorganic active compounds such as HOCl, peroxoacetic acid, quaternary ammonium, potassium peroxomonosulfate, chlorine dioxide, hydrogen peroxide, citric acid, lactic acid, dichloroisocyanurate, sodium hypochlorite or ethanol. However, these disinfectants can only be used in cleaning solutions or wiping solutions and have no permanent disinfecting effect.

Propiconazole

(±)-1-{[2-(2,4-Dichlorphenyl)-4-propyl-1,3-dioxolan-2-yl]methyl}-H-1,2,4-triazol (IUPAC),

Folpet

N-(trichloromethylthio)phthalimide,

Chlorocresols,

Fludioxonil

4-(2,2-difluoro-benzo[1,3]dioxo-4-yl)pyrrole-3-carbonitrile (IUPAC), and

Azoxystrobin

Methyl(E)-2-{2-[6-(2-cyanophenoxy)pyrimidin-4-yloxyl]phenyl}-3-methoxyacrylate (IUPAC)

are listed as approved protective agents for fibers, leather, rubber and polymerized materials in the “Helpdesk—Approved Agents—Federal Institute for Occupational Safety and Health”:
https://www.reach-clp-biozid-helpdesk.de/DE/Biozide/Wirkstoffe/Genehmigte-Wirkstoffe/Genehmigte-Wirkstoffe-0.html#PT9

However, these active ingredients are low molecular weight compounds, so there is always a risk that they will leach out of the protected materials.

Therefore, there have been numerous attempts to immobilize the disinfecting agents and pharmaceuticals to achieve a permanent disinfecting and/or pharmaceutically effective surface.

For example, international patent application WO 96/39821 discloses reagents and methods for modifying textiles with the aim of deactivating viruses on contact. To this end, textiles are modified by photochemically immobilizing hydrophilic polymers containing quaternary ammonium groups and hydrocarbon chains, resulting in a surface capable of disrupting lipid-coated viruses on contact. However, when these hydrophilic polymers are applied to nonwovens, there is no guarantee that they will be completely crosslinked by the light because partial areas will necessarily be shadowed. Consequently, a certain proportion of the polymers always remains soluble.

U.S. Pat. No. 5,883,155 discloses elastomeric films, wherein active chemicals such as biocides for medical purposes are uniformly dispersed in the form of gel inclusions. For example, the elastomeric film contains as active ingredients quaternary ammonium, phthalaldehyde, phenol derivatives, formalin, nonionic surfactants containing at least one polyoxyethylene block, hexamidine, iodine compounds, surfactants with virucidal activity, sodium and potassium dichromate, and hydrochlorites. However, these active ingredients are toxic and carcinogenic and are released into the environment.

U.S. Pat. No. 6,180,584 B1 discloses disinfecting mixtures with longer-lasting biocidal activity. The mixtures form an adhesive, transparent, water-insoluble polymer film on substrate surfaces that has a longer-lasting antimicrobial disinfecting effect. The effect lasts longer even without a new application. The surface disinfecting action is based on direct contact, and the ingredients are not released into a contacting solution in an amount that would disinfect the solution. The active ingredient is a metallic material, specifically silver iodide. However, this salt is photosensitive so that dark spots form in the mixture over time.

U.S. patent application 2007/0031512 A1 discloses that layered phyllosilicates are capable of adsorbing and/or binding viruses, thereby deactivating them. The layered phyllosilicates can be sprayed into human nostrils or can be included in a face mask to prevent infections. They can be suspended in water intended for skin contact to inactivate viruses or part of an HVAC filter that prevents the transfer of viruses from room to room, such as in a hospital. The phyllosilicates can be included in a paper or wipe to be used to deactivate viruses on furniture in hospitals and operating rooms and surgical equipment. In addition, the layered phyllosilicates can be used in paints for cleanrooms.

International patent application WO 2007/120509 discloses a mask comprising a plurality of layers, with the first layer comprising an acid or a salt or an ester of the acid. The second layer contains a base or a salt or an ester of the base. The third layer of the mask contains a metallic germicide selected from the group consisting of zinc, copper, nickel, iodine, manganese, tin, boron, silver and salts thereof, complexing agents and surfactants. Apparently, especially the third layer contains toxic substances.

U.S. patent application US 2007/0292486 A1 discloses biocidal polymer-nanoparticle/microparticle composites containing an ionic polymer and biocidal metal salts, in particular silver bromide. The silver bromide is uniformly distributed in the polymer matrix. It is assumed that the biocidal effect is due to silver particles releasing silver ions and bromide ions. It is also believed that the bromide ions make textiles flame retardant. The disadvantages of these composites are the high price of silver bromide, the photosensitivity of the salt, which causes dark discoloration in the composite layers, and the leaching of toxic silver ions. In addition, the biocidal metal salts are not concentrated on the surface of the composites, so most of the metal salts do not come into contact with the microorganisms.

International patent application WO 2008/127416 A2 discloses hydrophobic polymeric coatings that can be applied non-covalently to solid surfaces of metals, plastics, glass, polymers, textiles and other substrates such as fabrics, gauze, bandages, cloths and fibers in the same manner as paints by brush application, spraying or dipping to render the surface biocidal or bactericidal. The hydrophobic polymers contain quaternary ammonium groups with long chain aliphatic groups containing more than 10 carbon atoms. However, the hydrophobic polymers can be damaged by organic solvents and even removed from surfaces altogether, U.S. patent application US 2009/0081249 A1 discloses antimicrobial compositions containing two or more antiviral agents covalently bonded to a polymer. Suitable antiviral agents include sialic acid, zanamivir, oseltamivir, amantadine and rimantadine. The polymer is preferably water-soluble such as poly(isobutylene-alt-maleic anhydride), polyaspartic acid, poly(I-glutamic acid), chitosan, carboxymethylcellulose, carboxymethyldextran, or polyethyleneimine. The compositions can be prepared for enteral or parenteral administration. However, the antimicrobial compositions are difficult to produce on an industrial scale.

The American patent application US 2009/0320849 A1 discloses a face mask containing a filter material made of a fibrous substrate. The fibers thereof contain on their surface, in particular, a nonwoven fabric of polypropylene or polyester containing an acidic polymer, in particular of the polycarboxylic acid type. The face mask has an antiviral effect against inhaled or exhaled air. However, since the polycarboxylic acids such as polyacrylic acid are water soluble, they can be corroded by aqueous aerosols.

U.S. patent application US 2012/0016055 A1 discloses biocidal coating compositions comprising a biocide and non-ionic polymers and solvents.

The coating compositions form clear and non-tacky films and surfaces, but can be easily removed due to their solubility.

U.S. patent application US 2013/0344122 A1 discloses medical articles with antimicrobial properties and good barrier properties. The medical articles contain nonwoven fabrics made of polypropylene and a coating containing chlorhexidine acetate and trichlosan. These pharmaceuticals are dissolved in ethanol, for example, and sprayed onto the fabric until it is uniformly saturated. The fabric samples are then dried. The medical articles can be gowns, overshoes, drapes, wraps, caps, lab coats, and face masks. The disadvantage of these medical articles is that the pharmaceuticals are not firmly bound to the fibers of the nonwoven material and can be easily removed from it as dust or washed out by solvents.

International patent application WO 2014/149321 A1 discloses a coating with a reactive surface that has disinfecting and biocidal properties. The reactive compositions are renewable or “rechargeable” by reapplication of the active component and do not require removal, disposal or replacement. The reactive composition contains a hygroscopic polymer film such as cross-linked polyvinylpyrrolidone that has been treated with a liquid or gaseous oxidizing agent such as hydrogen peroxide, chlorine, peracetic acid, iodine, or mixtures thereof for a time such that the oxidizing agent has reacted with or is absorbed into the polymer film. The disadvantages of these reactive surface coatings are that they must be activated with toxic and corrosive or gaseous oxidizing agents, and these oxidizing agents are released back from the coatings.

U.S. patent application US 2014/0127517 A1 discloses films of linear or branched polyethylene mines with antiviral properties. These films are covalently bonded to surfaces and quaternized with hydrophobic side chains and modified with actinic radiation crosslinkable groups. The disadvantages of these films are that the polyethylenimines must be modified by polymer analogous reactions, After their application to surfaces, they must be irradiated with UV light. However, when applied to nonwoven materials, it cannot be ensured that all of the modified polyethyleneimine is reached and crosslinked by the UV radiation, International patent application WO 2016/116259 A1 discloses biocidal materials containing an organic polymer matrix or an inorganic ceramic matrix in which biocidal polyoxometalates are inhomogeneously distributed. Thus, the concentration of polyoxometalates may be higher at the surface of the matrices than in their interior.

U.S. Patent Application 2017/0275472 A1 discloses antimicrobial surface coating materials comprising (i) biocides such as chlorine dioxide, hydrogen peroxide, peroxyacids, alcohols, essential oils, antimicrobial components of essential oils, bleaching agents, antibiotics, phytochemicals, and mixtures thereof, (ii) inorganic-organic hollow bodies permeable to biocides, wherein the inorganic materials are metal oxides, metal complexes, metal salts, metal particles, and mixtures thereof, and the organic materials are non-ionic polymers such as polyethylene glycol or polyvinylpyrrolidone. The antimicrobial coating material is believed to have durable and versatile antimicrobial activity at high temperatures through contact killing, release, anti-adhesion and self-cleaning. The disadvantage is that volatile biocides are used, which are permanently released from the coating materials.

U.S. Pat. No. 10,227,495 B2 claims biocidal biopolymer coatings of crosslinked functionalized triglycerides and covalently bonded quaternary ammonium compounds. Crosslinking can be achieved by irradiation with actinic light or by polyisocyanates. A disadvantage is that the biocidal effect is limited to the use of one class of compounds, namely quaternary ammonium compounds.

Since the onset of the SARS-Co-V2 pandemic, numerous attempts have been made to develop new methods and materials to prevent the spread of the virus.

For example, A. J. Galante et al. of the Department of Industrial Engineering, University of Pittsburgh, and The Department of Ophthalmology, Charles T. Campbell Laboratory of Ophthalmic Microbiology, University of Pittsburgh, School of Medicine, describe superhemophobic anti-virofouling coatings for medical clothing (see also: SpecialChem. The material selection platform, Coating Ingredients, “Researchers Create New Washable, Textile, Coating the Can Repel Viruses.”, Published on 2020 May 26”; and “New coating could improve medical gear by making the Coronavirus slide right off.”; https://www.zmescience.com/science/news-science/coating-personal-protection-equipment-252342/). The resistant, multilayer coatings are made by sintering polytetrafluoroethylene (PTFE) nanoparticles in a solvent onto polypropylene microfibers. The disadvantage is that their production consumes a lot of energy and solvent as well as expensive PTFE, In addition, they do not kill viruses.

Researchers from Ben Gurion University, Israel, have developed nanoparticle-based coatings to prevent the spread of coronavirus. They have found that copper nanoparticles are most effective in this regard. The antiviral coatings can be brushed or sprayed onto surfaces, Common and well-known polymers containing nanoparticles of copper can be used. The nanoparticles enable the controlled release of metal ions onto the coated surfaces (see also SpecialChem. The material selection platform, Coating Ingredients, published on 2020 May 21). However, the copper nanoparticles and copper ions are toxic not only to microorganisms and viruses, but also to higher animals and humans.

Bio-Fence, Inc., Israel, has developed new antimicrobial coatings that are said to provide durable protection against coronaviruses. Apparently, the coatings contain a polymer that includes active chlorine that can be replenished by hydrochlorite solutions (see SpecialChem. The material selection platform, Coating Ingredients, published on 2020 May 12). The disadvantage of these coatings is the use of corrosive hypochlorite and active chlorine, which is believed to be bonded to nitrogen atoms in the form of >N—Cl groups. Thus, these materials are toxic and have an intense unpleasant odor.

On Jun. 30, 2020, the SpecialChem website published a note regarding “New Hybrid Coatings to Protect Interior Walls from Microbial Contamination” based on polyacrylates containing 3-(methacrylamino)propyltrimethylammonium chloride as a comonomer. The pendant quaternary ammonium groups act as biocidal centers:

https://physicsworld.com/a/cellular-nanosponges-could-neutralize-sars-cov-2/?utm_medium=email&utm_source=iop&utm_term=&utmcampaign=14258-46562&utm
content=Title%3A%20Cellular%20nanosponges%20could%20neutralize%20SARS-CoV-2%20%20-%20research update&Campaign+Owner=

A different approach is being taken at Concordia University, Canada, and the Canada-wide research network based at Concordia. This involves copper and titanium dioxide spray coatings designed to prevent the spread of Covid 19:

https://www.conordia.ca/content/shared/en/news/stories/2020/05/28/a-canada-wide-research-network-based-at-concordia-is-ready-to-make-work-surfaces-safer-for-frontline-staff.html

Still another approach is being taken by the TriOptoTec company of researchers at the University Hospitals of the University of Regensburg, Germany. (See “SpecialChem” 6/29/2020):

https://coatings.specialchem.com/news/industry-news/siegwerk-to-distribute-varcotec-antimicrobial-coating-innovation-000221983?lr=ipc20061570&li=200165733&utm_source=NL&utmmedium=EML&utm_cam_paign=ipc20061570&m_fNcWvxMSlgE4uDtFR2SUvnvKgGP6scLXvSt5WIN3KriGtDbdz%2BxzVTOAM%2BqiwO%2BV %2B21wdgflul3qOabGieKUHdikvRbBNp

The coating in question apparently contains dioctyl sodium sulfosuccinate in butyl diglycol. It also contains 10H-benzo[G]pteridine-2,4-dione derivatives (see U.S. Pat. No. 10,227,348 B2 and U.S. Pat. No. 9,796,715 B2) or phenalen-1-one derivatives (see U.S. Pat. No. 9,302,004 B2) as photosensitizers. The coating is mainly applied to paper or cardboard. When irradiated with visible light, the photosensitizer produces singlet oxygen, which kills the microorganisms on the surface of the paper or cardboard. The disadvantage is that this reaction only takes place in the light but not in the shade, so that numerous applications are excluded.

In the field of surface technology, it is generally known that a particularly strong lotus effect or superhydrophobicity can be achieved by a hierarchically structured surface design. For example, U.S. patent application US 2014/0238646 A1 discloses a method for producing hierarchically arranged structures of nanoscale inorganic phosphate particles homogeneously distributed on the surface of micrometer-scale phyllosilicate particles, Because these surfaces are not wetted and condensed moisture immediately forms droplets that roll off the surfaces, the contact time is too short for a biocidal or virucidal effect.

Aditya Kumar, Kalpita Nath and Poonam Chauhan of the Department of Chemical Engineering have developed a superhydrophobic antiviral coating with self-cleaning properties based on silver nanoparticles irradiated with UV radiation and then treated with perfluorodecyltriethoxysilane:

https://coatings.specialchem.com/news/industry-news/new-superhydrophobic-antiviral-coating-self-cleaning-properties-000221961?lr=ipc20061569&li=200165733&utm_source=NL&utmmedium=EML&utmcampaign=ipc20061569&mi=owCobZJ7BFfD70L%2BHEhcWDRZOrH4AKAPPd55vGetHRPb8itAEOFCfeJRcddTTWGNwEzLArkBh9IQcRenmqXfr87TriboV
https://www.technicaltextile.net/news/iit-ism-s-silver-nanooarticle-anti-viral-textile-coating-268082.html

(Cf. also SpecialChem for Coatings, Industry News, 6/23/2020), However, because of the superhydrophobicity, the disadvantages described above are also to be expected here.

Still another approach is being taken by J. Mostaghimi of the University of Toronto, Canada. See “SpecialChem The material selection platform, Sep. 7, 2020”, Twin-wire Arc Spray Technology to Deposit Cu on Fabrics):

https://coatings.specialchem.com/news/industry-news/new-way-cu-coatings-masks-covid19-transmission-000222593?lr=ipc20091591&li=200165733&utm_source=NL&utmmedium=EML&utm campaign=ipc20091591&mi=LKHowSxEDOOgaf6IRKgtvs82qOFcOeUHDQgfVqznX2JvZxquM0NNdHnP8q95KPhnJ0ofLb9h8b0_4U4K4q4szQnptKr2LD&status=valid
and “Anti-viral copper coatings could help slow the transmission of COVID 19,” Department of Mechanical & Industrial Engineering, University of Toronto, lynsev@bmie.utoronto.ca: Aug. 31, 2020.

Jinghzi Pu et al. from the School of Science at IUBUI, Indiana, USA, developed mask filters that mimic the internal structure of fish gills. The complex structures are fabricated by 3D printing and then coating the surface with copper by electroplating. By increasing the surface area over which air passes, the biocidal effect of the copper is said to increase. The developers speculate that these structures could also be suitable for filters for air-conditioning systems in buildings and aircraft (see SpecialChem, Industry News, Researchers Use Cu Coating on Plastic Mask Filters to Reduce Virus Spread, Publ. Sep. 17, 2020

https://coatings.specialchem.com/news/industry-news/cur-coating-plastic-mask-filters-reduce-virus-spread-000222707?lr-ipc20091594&li=200165733&utmsource=NL&utmmedium=EML&utmcampagin=ipc20091594&mi=fM1fEM0ZwQ1A6LPH0ipPWKoH2EusD4Rm30xmwWHtDbV2rPu33i6wC0fu0%2Bwv4Rm1vNWcGxaS2K9FxoWaHR7r3%2B8aWffp&status=valid cf, also
https://news.iu.edu/stories/2020/09/iupui/releases/16-copper-coating-3d-printed-plastic-fillers-pandemic-fighter.html).

It is to be feared, however, that at a high air flow rate with high flow velocities, these complex structures generate intense noise such as hissing or whistling.

Air cleaners are mobile devices for cleaning air with the help of filters.

According to their separation efficiency, the filters can be divided into
high-performance particulate filter (EPA=Efficient Particulate Air filter), smallest filterable particle size; 100 nm,
HEPA (High Efficiency Particulate Air filter), smallest filterable particle size; 100 nm,
high-performance HEPA filter (ULPA=Ultra Low Penetration Air filter), smallest filterable particle size: 50 nm,
medium filter, smallest filterable particle size; 300 nm,
prefilter, smallest filterable particle size: 1000 nm, and
automotive cabin filter, smallest filterable particle size: 500 nm Thus, no filters are available for the range from 1 nm to 50 nm.

Depending on the particle size, their filtering effect is based on the following effects;
Diffusion effect; Very small particles (particle size: 50 nm to 100 nm) do not follow the gas flow, but have a trajectory similar to Brownian motion due to their collisions with the gas molecules and thus collide with the filter fibers, to which they adhere. This effect is also called diffusion regime.
Barrier effect: Smaller particles (particle size: 100 nm to 500 nm) that follow the gas flow around the fiber stick if they get too close to the filter phase. This effect is also called interception regime.
Inertial effect: Larger particles (particle size: 500 nm to >1 μm) do not follow the gas flow around the fiber, but bounce against it due to their inertia and stick to it. This effect is also referred to as inertial impaction and interception regime.

In the particle size range from 100 nm to 500 nm, the diffusion effect and the barrier effect occur together. In the particle size range from 500 nm to >1 μm, the inertial effect and the barrier effect also occur together.

According to the filter effects, particles of a particle size of 200 nm to 400 nm are the most difficult to separate. They are also referred to as MMPS most penetrating particle size. The filter efficiency drops to 50% in this size range. Larger and smaller particles are better separated due to their physical properties.

One classifies EPA, HEPA, and UPLA according to effectiveness for these grain sizes using a test aerosol of di-2-ethylhexyl-sebacate (DEHS). K. W, Lee and B. Y, H. Liu, in their article “On the Minimum Efficiency and the Most Penetrating Particle Size for Fibrous Filters” in Journal of the Air Pollution Control Association, Vol. 30, No. 4, April 1980, pages 377 to 381, give formulas that allow the smallest efficiency and MMPS to be calculated for fibrous filters due to the diffusion effect and the inertia effect. The results show that MMPS decreases with increasing filtration speed and with increasing fiber volume fraction and increases with increasing fiber size.

Particularly critical, however, is the fact that there are no filters for nanoparticles of an average particle size d₅₀ of 1 nm to <50 nm. It is precisely these particles that are easily deposited in bronchi and alveoli and generally have the highest mortality and toxicity. They can therefore cause diseases such as asthma, bronchitis, arteriosclerosis, arrhythmia, dermatitis, autoimmune diseases, cancer, Crohn's disease or organ failure.

This is especially critical because the depth filters or HEPA filters are used in medical applications such as operating rooms, intensive care units and laboratories, as well as in clean rooms, nuclear technology and air scrubbers, among others.

Another technology problematic in this respect are electrostatic precipitators for electric gas cleaning, electro-dust filters or electrostatic precipitators, which are based on the separation of particles from gases by means of the electrostatic principle, Separation in the electrostatic precipitator can take place in five separate phases:
1. Release of electrical charges, mostly electrons,
2, Charging of the dust particles in the electric field or ionizer,
3. Transport of the charged dust particles to the collecting electrode,
4. Adhesion of the dust particles to the collecting electrode and
5. Removal of the dust layer from the collecting electrode.

However, it is not possible to completely separate particles in the nanometer range, so there is a risk of contamination with respirable particles in the vicinity of such systems.

These electrostatic precipitators are often used in exhaust gas treatment. In this process, amines, carbon dioxide, ammonia, hydrochloric acid, hydrogen sulfide and other toxic gases are removed from the exhaust gas stream with the aid of membranes. Since the electrostatic precipitators cannot completely remove the finest particles, these damage the membranes and reduce their separation efficiency.

For details concerning toxicology, reference is made to the review articles by Günter Oberdörster, Eva Oberdörster, and Jan Oberdörster, “Nanotoxicology. An Emerging Discipline Evolving from Studies of Ultrafine Particles,” in Environmental Health Perspectives Volume 113. (7), 2005, 823839, and Günter Oberdörster, Vicki Stone, and Ken Donaldson, “Toxicology of nanoparticles: A historical perspective,” Nanotoxicology, March 2007; 1(1): 2-25.

Viruses present outside cells are scientifically called virions. They range in diameter from 15 nm to 440 nm and are significantly smaller than bacteria, most of which range in diameter from 1 μm to 5 μm. The viruses or virions thus have sizes that fall into the “filter gaps” of 1 nm to 50 nm and from 200 nm to 400 nm.

Therefore, the air purifiers equipped with filters can at best reduce the concentration of viruses or virions in the indoor air, but they cannot completely remove or destroy them because they lack a disinfecting effect.

Non-sedimenting aerosols generated by humans and animals, especially aerosols generated by breathing, coughing or sneezing, which disperse very rapidly in large volumes in enclosed spaces, play a central role in the transmission of viruses from human to human, from animal to human, from animal to animal and from human to animal. They contribute significantly to the spread of disease. Since the non-sedimenting aerosols generally have a particle diameter of 0.1 nm to 100 nm, they can only be intercepted incompletely, if at all, by the air filters.

Therefore, if one intends to keep the concentration of viruses and virions in the air in closed rooms below a threshold above which there is a high risk of infection, the air must be permanently circulated in large quantities. However, this currently only works with stationary air-conditioning systems, but these often cannot be retrofitted into buildings, means of transport, etc. Another disadvantage is that powerful air conditioners, fans and mobile air cleaners often make a loud noise, which is perceived as annoying. Their particularly strong air currents often cause health problems such as colds and joint pain.

In the company magazine of “LUFTREINIGERDEPOT Your specialist for healthy indoor air”,

https://www.luftreinigerdepot.de/gegen/bakterien-und-viren?=1&o=2&n=20&t=784
downloaded on Aug. 25, 2020, the problems are once again made clear (original quote at the beginning):
“Air purifiers against viruses & bacteria—also against the coronavirus?
An air purifier against bacteria and viruses is particularly useful for waiting rooms of doctors' offices, for offices or packages, for other public spaces such as canteens, hair salons or nail salons. Wherever people come together and the air is more or less stagnant, the risk of infection increases, which can be reduced by the use of air purifiers. Whether air purifiers are effective against the Covid 19 coronavirus has not yet been tested due to the fact that it has not been around for long. Since it is not a completely new virus, but a mutated form of already known viruses, much speaks in favor of a respective effectiveness.
Find out which air purifiers are particularly good against bacteria and viruses below.

### Important Notice:

Please note that although air purifiers can significantly reduce the concentration of viruses and bacteria in the air, they cannot completely prevent it. The best protection against coronavirus is avoidance of social contact and frequent and thorough hand washing. In any case, please follow the requirements of the federal and state governments.
“Due to the small size of bacteria and viruses, air purifiers must have the correct filters to safely capture airborne hazards. Air purifiers with HEPA filters work very effectively against microscopic foci of infection, safely filtering even particularly tiny bacteria with a particle size as small as 0.3 micrometers (μm) from indoor air. Additional methods such as photocatalytic filters, nano-silver filters or added ionizers can help to further improve the efficiency of air purifiers. In order to trap bacteria and viruses as quickly as possible in the available filters, a high air flow rate is also important for air purifiers. An air purifier should be able to clean the entire room air at least twice per hour. Manufacturers of premium devices target complete cleaning of room air up to 5 times per hour.
(original quote end).

It is hoped that the effect of fans will be improved by the co-application of UVC radiation of a wavelength λ of 280 nm to 100 nm. In contrast, SARS-Co-V2 viruses and virions have a diameter of 60 to 140 nm, which is partly smaller than the wavelength λ of UVC radiation, Thus, the interaction between UVC radiation and SARS-Co-V2 viruses and virions is weak at best, and the claims that UVC radiation causes complete killing of these pathogenic virions and viruses must be doubted.

In the international patent application WO 2020/078577 A1 it is proposed to use acoustophoresis devices combined with filters to destroy microorganisms. Further details or whether this method is also suitable for viruses and virions are not given.

Prions are proteins that can exist in the animal organism in both physiological (normal) and pathogenic (harmful) conformations (structures). They do not proliferate by division, but by induced modification of neighboring molecules. Recent studies confirm that prions are also transmitted via the air and aerosols:

Study confirms: Prions can be transmitted via the air | News | CORDIS | European Commission (eurooa.eu)

### (Downloaded Oct. 20, 2021).

Since no curative treatment of prion diseases is yet possible, it is desirable to at least block this transmission pathway, Allergens are substances that can trigger hypersensitivity reactions or allergic reactions via mediation of the immune system. The different hypersensitivity reactions are allergies, pseudoallergies and intolerances. Allergens are antigens and have no chemical commonality. Therefore, it is not possible to develop compounds that destroy allergens. Numerous allergens and pseudoallergens, i.e. non-allergenic irritants, are often airborne. Examples of such pseudoallergens include particulate matter, aerosols from adhesives, cleaning agents, and sprays, perfumes, tobacco smoke, and combustion products from candles and incense. Here, too, it would be highly desirable to block the transmission route via air and aerosols.

Whether the methods and devices described above can provide protection against prions, allergens and pseudoallergens transmitted by air and aerosols is doubtful.

A millennia-old method of immunization against smallpox is variolation. The contents of the pustules of smallpox or chickenpox were transmitted from person to person by inoculation. In other words, a live attenuated vaccine consisting of attenuated viruses was applied.

Another way to reduce the concentration of virions and aerosols in closed rooms is intensive shock ventilation by outside air. This requires, first, that rooms be located on the outside of buildings so that such ventilation opportunities are available at all and, if so, that weather conditions permit ventilation. This is likely to be difficult in low outdoor temperatures, driving rain, thunderstorms, hail, snow, freezing rain, etc.

### Object of the Present Invention

The present invention was based on the object of finding a device with which microorganisms, viruses, virions, prions, allergens and pseudoallergens can be safely and completely attenuated and/or killed and/or their transmission pathways via the air blocked, With the aid of the device, the attenuation and/or the killing and/or the blocking of the transmission pathways of the microorganisms, virions, virions, prions, allergens and pseudoallergens is to be carried out in a simple manner without the need for permanently circulating and/or exchanging large quantities of room air, in order to bring the content of aerosols with microorganisms, viruses and virions or of microorganisms, viruses, virions, prions, allergens and pseudoallergens in the air itself to a level which prevents infections, allergies and asthma, and also to keep it at this level. This is also intended to provide significant energy savings over conventional prior art fans, which require at least five times the air exchange per hour. Last but not least, the devices are said to be considerably smaller the conventional air filters and yet more effective than them.

The devices should be applicable to humans and animals.

### Solution According to the Invention

Accordingly, the device according to the invention for attenuating and/or killing and/or chemically and/or physically chemically modifying microorganisms, in particular pathogenic microorganisms, viruses, virions, prions, allergens and pseudoallergens and/or their residues and/or decomposition products and/or for blocking the transmission pathways thereof according to independent patent claim 1 was found, hereinafter referred to as the “device according to the invention”.

Advantageous embodiments of the device according to the invention are the subject matter of dependent claims 2 to 11.

Furthermore, the method according to the invention was found for attenuating and/or killing and/or chemically and/or physically chemically modifying microorganisms, in particular pathogenic microorganisms, viruses, virions, prions, allergens and pseudoallergens and/or their residues and/or decomposition products and/or for blocking their transmission pathways according to the independent claim 12, which is hereinafter referred to as the “method according to the invention”. A preferred embodiment of the method according to the invention is the subject matter of dependent claims 13 and 14.

Last but not least, the use of the device and method according to the invention according to claim 15 was found, hereinafter referred to as the “use according to the invention”, Advantages of the invention With regard to the prior art, it was surprising and not foreseeable for the person skilled in the art that the object on which the present invention was based could be solved with the aid of the device according to the invention, the method according to the invention and the use according to the invention without the disadvantages of the prior art occurring.

In Particular, it was Surprising that with the Aid of the Method and Device according to the invention, microorganisms, viruses, virions, prions, allergens and pseudoallergens that are freely suspended or contained in aerosols could be reliably and completely attenuated and/or killed and/or their transmission pathways via the air blocked. With the aid of the device, the attenuation and/or killing of microorganisms, in particular pathogenic microorganisms, viruses, virions, prions, allergens and pseudoallergens, and/or the blocking of their transmission pathways could be carried out in a simple manner without having to permanently circulate and/or exchange large quantities of room air, in order to reduce the content of aerosols in the air containing microorganisms, in particular pathogenic microorganisms, viruses, virions, prions, allergens and pseudoallergens, or free-floating microorganisms, in particular pathogenic microorganisms, viruses, virions, prions, allergens and pseudoallergens, themselves to a content that prevents infections, allergies and asthma, and also to maintain them at this content. This also provided significant energy savings over conventional prior art air filters, which require at least five times the air exchange per hour, Last but not least, the devices were considerably smaller than conventional air filters and yet more effective than them.

Furthermore, it was surprisingly found that allergy sufferers experienced a significant reduction in allergic reactions and desensitization in room air purified according to the invention. There was a significant alleviation of symptoms for sufferers from asthma.

Surprisingly, the device and method according to the invention could also be applied in animal husbandry.

Further advantages can be seen in the following description.

### DETAILED DESCRIPTION OF THE INVENTION

The device according to the invention is shielded against the emission of actinic radiation so that it can be used safely in living rooms and offices.

The device according to the invention can be arranged vertically, obliquely or horizontally in space. Its outer wall may have a circular, oval, elliptical, quadrangular, pentagonal, hexagonal or octagonal outline, A circular outline is particularly preferred, so that the entire device according to the invention is drum-shaped.

The device according to the invention is constructed of materials that are stable and/or stabilized against UVC light radiation. Suitable UVC-stable materials are metals such as steel, stainless steel and in particular anodized aluminum, glasses, metal-coated plastics or with UV absorbers such as benzotriazoles, hydroxyphenyltriazines, hydroxybenzophenones, oxalanilides, sterically hindered amines (HALS), titanium dioxide, iron oxide pigments, zinc oxide and stearates of lead, cadmium, tin, barium, calcium, aluminum and/or zinc.

The device according to the invention comprises at least one intake region shielding the actinic radiation and having at least one intake opening for the sucked-in microorganisms, in particular pathogenic microorganisms, viruses, virions, prions, allergens and pseudoallergens of all kinds, floating freely or on and in aerosols.

The shielding of the emissions of actinic radiation in the at least one intake opening is accomplished with the aid of at least two, preferably at least three, particularly preferably at least four, and in particular at least five grids, perforated plates, perforated screens and lamellae arrangements made of metals, metal-coated plastics and window glass, which are arranged in a staggered manner and which are permeable to air, by macroporous carbon sponges and/or macroporous glass frits.

In another embodiment, shielding of actinic radiation emissions in the intake region is achieved by at least one plate with vertically aligned, parallel, zigzag-shaped channels. The channels may have a circular, oval, triangular, quadrangular or polygonal cross-section. Their clear width can vary widely and be optimally adapted to the respective requirements, Preferably, the clear width is 1 μm to 2 mm. Its length, measured along the zigzag lines, is preferably 10 mm to 400 mm.

The at least one intake region is connected to at least one air conveying region at a circumferential separation point in such a way that it can be detached. The connection is made by bayonet connections, screw connections, flange connections and/or plug connections. The other areas of the device according to the invention are connected in the same way.

According to the invention, the air conveying region comprises at least one support for at least one axial rotor, fan or ventilator driven by an electric motor with speed control and having at least two rotor blades.

Examples of suitable fans or blowers are axial fans such as the well-known Papst fans from ebm-papst Mulfingen GmbH & Co. KG, The axial fans can be arranged next to each other and/or in series behind each other to increase the suction and pressure effect, Preferably, the EC centrifugal modules—RadiCal® from ebm-papst Mulfingen GmbH & Co. KG are used. The axial fans can be equipped with devices for volume flow measurement via differential pressure gauges or a U-fluid column. These can control and visualize the current volume flow during operation in the corresponding suction or pressure range.

Alternatively or additionally, at least one fan adapted to the dimensions of the device according to the invention can be used.

Advantageously, the air conveying region contains at least one controllable device for heating or cooling the intake air, such as Peltier elements or electric heating coils.

At a further separation point, at least one irradiation region with at least one radiation source for actinic (effective) radiation is detachably connected.

Actinic radiation can be corpuscular radiation, such as electron radiation, proton radiation, alpha radiation, positron radiation, and beta radiation, as well as electromagnetic radiation, such as microwave radiation, infrared radiation, blue light, UVA, UVB and UVC radiation, X-rays, or gamma radiation.

In particular, blue light and/or UVC radiation, but especially UVC radiation, is used as actinic radiation.

In particular, common and well-known UVC emitters, such as those used for disinfecting aquariums and ponds, can be considered as UVC radiation sources. These emit UVC radiation of a wavelength around 240 nm, wherein the wavelength at 185 nm, which is responsible for the generation of ozone, is not emitted.

The at least one irradiation region advantageously comprises at least three, in particular at least four, support rods extending parallel to the at least one radiation source for in each case at least two, preferably at least three, particularly preferably at least four and in particular at least five pairs of (i) planar metal rings lying parallel one above the other and reaching close to the outside of the at least one radiation source (4.1), each having a circumferential air passage between the outer edge and the inner wall of the irradiation region, and (ii) planar, horizontal metal rings flush with the inner wall of the irradiation region and extending as far as close to the outer side of the at least one radiation source, wherein the at least three parallel support rods are anchored to or in the at least one holder.

The at least one radiation source is connected in at least one power supply region to at least one controllable power supply. If necessary, the power supply can be regulated down to 0.0 volts during operation of the device according to the invention. The at least one power supply is attached to the power supply region by at least one circumferential, planar holder having power leads. The at least one holder has openings for the UVC-treated air to enter into at least one acoustophoresis region, which is detachably connected to the power supply region of another separation point as described above.

The at least one acoustophoresis region includes, for generating at least one stationary acoustic ultrasonic field, at least one acoustophoresis device having at least one wall-free flow region and/or having at least one flow tube with a closed wall enclosing at least one flow channel. The at least one flow channel is used for the flow of the irradiated air.

The at least one wall-free flow region is enclosed by at least two, preferably at least three, preferably at least four, particularly preferably at least five, and in particular at least six pairs of mutually associated and opposing ultrasonic emitters or ultrasonic emitter-receivers and/or by at least two, preferably at least three, preferably at least four, particularly preferably at least five, and in particular at least six pairs, each of an ultrasonic emitter or ultrasonic emitter-receiver and a respective reflector associated therewith and opposing it.

Alternatively or additionally, at least two, preferably at least three, more preferably at least four, particularly preferably at least five, and in particular at least six ultrasonic emitters and/or ultrasonic emitter-receivers are arranged centrally in the at least one wall-free flow region. The ultrasonic waves are selected from the group consisting of standing, modulated and non-modulated longitudinal waves and transverse waves.

In the at least one closed wall of the at least one flow tube, on the outside and/or the inside and/or in the respective closed wall itself, there are arranged at least two, preferably at least three, more preferably at least four, particularly preferably at least five and in particular at least six pairs of mutually associated and mutually opposite ultrasonic emitters or ultrasonic emitter-receivers and/or at least two, preferably at least three, more preferably at least four, particularly preferably at least five and in particular at least six pairs of in each case one ultrasonic emitter or ultrasonic emitter-receiver and in each case one opposite reflector associated therewith.

Alternatively or additionally, at least two, preferably at least three, more preferably at least four, particularly preferably at least five, and in particular at least six ultrasonic emitters and/or ultrasonic emitter-receivers are arranged centrally in the at least one wall-free flow region. The ultrasonic waves are selected from the group consisting of standing, modulated and non-modulated longitudinal waves and transverse waves.

The respective at least two, preferably at least three, more preferably at least four, particularly preferably at least five and in particular at least six pairs described above are arranged one behind the other, as seen in the direction of flow, or are arranged in such a way that the imaginary connecting lines between the respective at least two, preferably at least three, more preferably at least four, particularly preferably at least five and in particular at least six pairs cross at an angle of 90°.

Preferably, the ultrasonic waves have a frequency of 1 kHz to 800 MHz. The at least one stationary acoustic ultrasonic field has an energy input of 0.25 W to 1 kW at a power level of 40 to 250 dB.

Preferably, the ultrasonic emitters are selected from the group consisting of loudspeakers, vibrating diaphragms, piezoelectric loudspeakers, acoustic transducers, virtual sound sources, immersion coils, magnetostatic loudspeakers, ribbon, foil and jet tweeters, horn drivers, bending wave transducers, plasma loudspeakers, electromagnetic loudspeakers, exciters, ultrasonic transducers and phantom sound sources.

The sound pressure of the ultrasonic waves emitted by the ultrasonic emitters is preferably adjusted so that the emitted particles and/or fragments of microorganisms, in particular pathogenic microorganisms, viruses, virions, prions, allergens and pseudoallergens have an average molecular weight of 5 kDa to 10 kDa, preferably 6 Da to 9 kDa, particularly preferably 6.5 kDa to 77 kDa and especially 6.7 kDa to 7.3 kDa.

The at least one acoustophoresis device can be heated or cooled using suitable devices such as Peltier elements.

The at least one acoustophoresis device has at least one air outlet for discharging the acoustophoretically treated air).

The at least one acoustophoresis device is enclosed by circumferential electronics protected by planar shielding for controlling the at least one acoustophoresis device, the at least one axial rotor or fan, and the at least one irradiation region. In particular, the electronics are used to generate, monitor, and stabilize at least one feedback loop for adjusting and stabilizing the steady-state acoustic ultrasonic field.

Preferably on the outer wall of the acoustophoresis region, switches, controllers, sockets for power connections, function lights and LED indicators for the air flow, the air temperature, the speed of the axial rotor or fan and the sound pressure in the ultrasonic field are arranged. The device according to the invention may also contain at least one powerful rechargeable battery so that the operation of the device can continue, for example, in the event of a change of location or in the absence of a power source.

At at least one further separation point, the acoustophoresis region is detachably connected, as described above, to at least one air outlet region shielding the actinic radiation. In this at least one air outlet region, preferably the same air-permeable UVC shields are used as in the at least one intake region for the contaminated air.

At at least one further separation point, the air outlet region shielding the actinic radiation is detachably connected to at least one air outlet region for the attenuated and/or killed microorganisms, in particular pathogenic microorganisms, viruses and virions as well as chemically and/or physicochemically modified, deactivated prions, allergens and pseudoallergens containing air irradiated with actinic radiation and treated acoustophoretically. The treated air discharged into the environment generally no longer contains aerosols, as these are destroyed during the acoustophoretic treatment.

In a further embodiment of the device according to the invention, the device according to the invention as described above is “turned upside down”, so to speak. This means that the contaminated air is first passed through the at least one acoustophoresis region described above and then through the at least one irradiation region and then discharged into the room air.

In still another embodiment of the device according to the invention, the contaminated air is first passed through the at least one irradiation region, then through the at least one acoustophoresis region, and finally again through at least one irradiation region, and then discharged into the room air.

In a fourth embodiment of the device according to the invention, the contaminated air is first passed through the at least one irradiation region, then through the at least one acoustophoresis region, then again through at least one irradiation region and finally through at least one further acoustophoresis region and then discharged into the room air.

In a fifth embodiment of the device according to the invention, the contaminated air is first passed through at least one acoustophoresis region, then through at least one irradiation region, then again through at least one acoustophoresis region and finally through at least one further irradiation region and then discharged into the room air.

If required, at least one device for filtration selected from the group consisting of non-biocide and/or biocide coated EPA, HEPA, ULPA, medium and activated carbon filters may be provided downstream of the at least one air outlet region.

The biocidal coatings may contain the biocides listed in the “Helpdesk-Approved Agents—Federal Institute for Occupational Safety and Health”:

https://www.reach-clp-biozid-helpdesk.de/DE/Biozide/Wirkstoffe/Genehmigte-Wirkstoffe/Genehmilgte-Wirkstoffe-0.html#PT9

The

“List N: Products with Emerging Viral Pathogens AND Human Coronavirus Claims for Use against SARS-CoV-2, Date Accessed: 05/31/2020 of the EPA lists, US Govt.”
lists numerous organic and inorganic active compounds such as HOC, peroxoacetic acid, quaternary ammonium, potassium peroxomonosulfate, chlorine dioxide, hydrogen peroxide, citric acid, lactic acid, dichloroisocyanurate, sodium hypochlorite or ethanol, which can be used if they can be immobilized and/or bound in the sense of slow release. Furthermore, the known ionic liquids can be considered, since they have practically no vapor pressure. In principle, ionic liquids are molten salts with a low melting point. They include not only those that are liquid at ambient temperature, but also all salt compounds that melt preferably below 150° C., more preferably below 130° C. and especially below 100° C., In contrast to conventional inorganic salts such as common salt (melting point 808° C.), lattice energy and symmetry are reduced in ionic liquids due to charge delocalization, which can lead to solidification points as low as −80° C. and below, Due to the numerous possible combinations of anions and cations, ionic liquids with very different properties can be produced (cf. a. Römpp Online 2020, “ionic liquids”). All cations commonly used in ionic liquids can be considered as organic cations, Preferably, they are non-cyclic or heterocyclic onium compounds. Preferred are non-cyclic and heterocyclic onium compounds selected from the group consisting of quaternary ammonium, oxonium, sulfonium and phosphonium cations, as well as uronium, thiouronium and guanidinium cations in which the single positive charge is delocalized over several heteroatoms, Quaternary ammonium cations are particularly preferred, and heterocyclic quaternary ammonium cations are more particularly preferred.

Last but not least, the biocidal polyoxometallates POM described in detail on page 13, line 15, to page 32, line 27, in international patent application WO 2016/116259 A1 are also considered.

Preferably, the method according to the invention is carried out with the device according to the invention. The method according to the invention comprises at least the following method steps:
(A) Suction of air containing microorganisms, in particular pathogenic microorganisms, viruses, virions, prions, allergens and pseudoallergens and/or air containing aerosols with microorganisms, in particular pathogenic microorganisms, viruses, virions, prions, allergens and pseudoallergens through the at least one intake opening of at least one air-permeable intake region shielding the actinic radiation;
(B) conveying the air through at least one air conveying region with the aid of at least one axial rotor with speed control or at least one controllable fan into at least one irradiation region;
(C) attenuation and/or killing and/or chemically and/or physicochemical modification of the microorganisms contained in the air, in particular pathogenic microorganisms, viruses, virions, prions, allergens and pseudoallergens, by irradiation of the air in the at least one irradiation region with the actinic radiation from at least one radiation source,
(D) entry of the resulting air treated with actinic radiation through at least one opening into at least one acoustophoresis region with at least one acoustophoresis device,
(E) generating at least one controllable, stationary, acoustic ultrasonic field in the at least one acoustophoresis device for acoustophoretic treatment of the air flowing through,
(F) acoustophoretic treatment of the aerosols, attenuated and/or killed and/or chemically and/or physicochemically modified microorganisms, in particular pathogenic microorganisms, viruses, virions, prions, allergens and pseudoallergens and/or their residues and/or decomposition products contained in the air flowing through in the controllable, stationary, acoustic ultrasonic field for the generation of acoustophoretically treated air,
(G) discharging the acoustophoretically treated air from at least one air outlet of the at least one acoustophoresis device through at least one air outlet region shielding the actinic radiation, and
(H) discharging the air treated with actinic radiation and acoustophoretically from the at least one air outlet region directly to the environment or via at least one downstream device for filtration.

As mentioned above, blue light and/or UVC radiation, but especially UVC radiation, is preferably used as actinic radiation.

The device according to the invention and the method according to the invention are excellently suited for use according to the invention. In particular, they are suitable for the attenuation and/or killing and/or the chemical and/or physicochemical modification of free microorganisms and/or microorganisms bound in and/or to aerosols, in particular pathogenic microorganisms, viruses, virions, prions, allergens and pseudoallergens and/or their residues and/or decomposition products in the air, especially in living rooms, sickrooms, operating theaters, treatment rooms in medical practices and physiotherapeutic facilities, laboratories of all kinds, inns, restaurants, bistros, hotel rooms, schoolrooms, classrooms, gyms, trains, cars, buses, cabs, caravans, mobile homes, camping tents, airplanes, ship cabins, offices, conference rooms, meeting rooms, theaters, cinemas, ship terminals, railroad stations, airport terminals, elevators, workshops, factory halls, stairwells, stores, and animal pens.

The attenuated and/or killed microorganisms, in particular pathogenic microorganisms, viruses and virions, as well as their fragments and decomposition products contained in the room air, no longer pose a risk of infection. By treating the prions, allergens and pseudoallergens with UVC and/or acoustophoresis, their transmission pathways are blocked, Without being bound to a theory, it is believed that the attenuated and/or killed microorganisms, viruses and virions, as well as their fragments and decomposition products, appear to strengthen the immune system. The prions, allergens and pseudoallergens are chemically and/or physicochemically modified by the treatment according to the invention to such an extent that they no longer cause harm to humans and animals. Therefore, the device according to the invention and the method according to the invention can also be excellently applied in animal husbandry.

### BRIEF DESCRIPTION OF THE FIGURES

FIGS. 1 and 2 serve to illustrate the structure of the device according to the invention and its mode of operation. They are therefore not drawn to scale, but emphasize their essential features. They are also to be understood only as exemplary and not as restrictive, wherein:

FIG. 1 shows the side view of the drum-shaped device 1 according to the invention and

FIG. 2 shows the top view of the vertical longitudinal section along the central axis of the drum-shaped device 1 according to the invention.

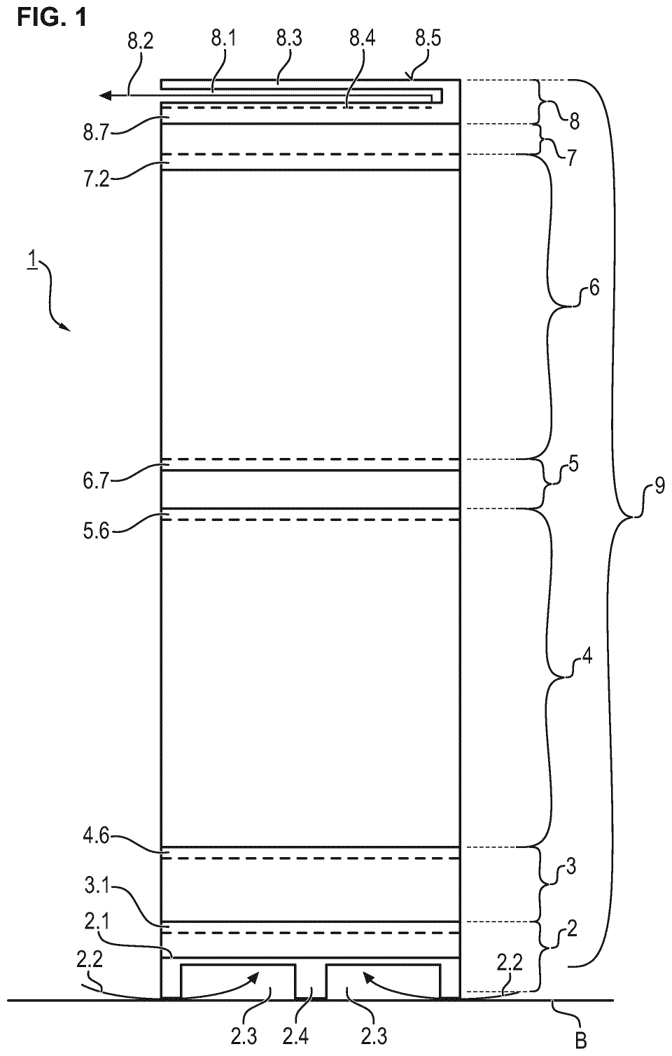

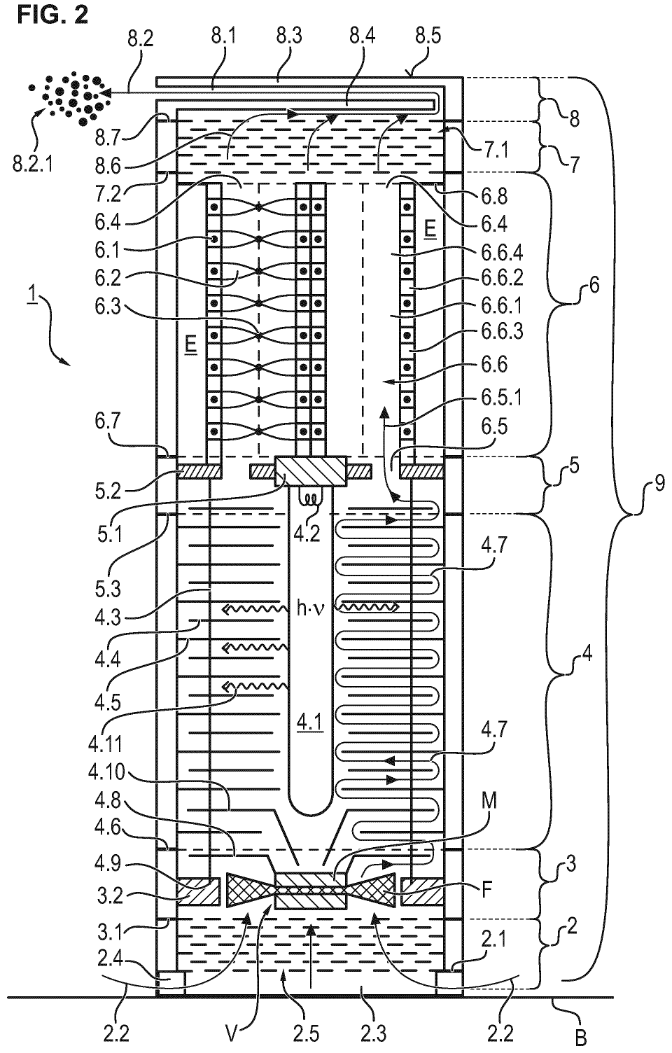

In FIGS. 1 and 2, the reference signs have the following meaning:
1 Drum-shaped device 1 according to the invention
2 Tubular intake region
2.1 Circumferential separation point between the feet 2.4 and the air-permeable grid arrangement 2.5 shielding the UVC radiation
2.2 Sucked-in contaminated air
2.3 Intake opening with circular circumference
2.4 Foot
2.5 UVC-shielding, air-permeable grid arrangement
3 Tubular air conveying region
3.1 Circumferential separation point between the air conveying region 3 and the intake region 2
3.2 Holder of the horizontally mounted axial rotor or fan V
4 Tubular UVC region
4.1 UVC light source
4.2 Filament
4.3 Vertical support rod
4.4 Planar horizontal metal ring with circumferential air passage between the outer edge and the inner wall of the tubular UVC region 4
4.5 Planar horizontal metal ring flush with the inner wall of the tubular UVC region 4.
4.6 Circumferential separation point between the tubular UVC region 4 and the tubular air conveying region 3
4.7 Guided air flow
4.8 First angled air guide ring
4.9 Detachable anchoring of the vertical support rod 4.3 to the holder 3.2
4.10 Second angled air guide ring
4.11 UVC light irradiated between the horizontal metal rings 4.4 and 4.5
5 Tubular power supply region
5.1 Holder and power supply for the UVC light source 4.1
5.3 Annular holder of the power supply 5.1 with power line and air passages 6.5
5.4 Circumferential separation point between the tubular UVC region 4 and the tubular power supply section
6 Tubular acoustophoresis region
6.1 Ultrasonic emitter, reflector
6.2 Wave belly of a standing ultrasonic wave
6.3 Wave node of a standing ultrasonic wave
6.4 Air outlet from the tubular acoustophoresis device 6.6
6.5 Opening for the entry of UVC-treated air 6.5.1 into the tubular acoustophoresis device 6.6
6.5.1 Air irradiated with UVC radiation
6.6 Tubular acoustophoresis device
6.6.1 Wall-free flow region
6.6.2 Flow tube
6.6.3 Closed wall
6.6.4 Flow channel
6.7 Circumferential separation point between the acoustophoresis region 6 and the UVC region 5.
6.8 Circumferential, planar, horizontal shielding for electronics E
7 Tubular air outlet region
7.1 UVC-shielding, air-permeable grid arrangement
7.2 Circumferential separation point between the acoustophoresis region 6 and the air outlet region 7
8 Planar horizontal air outlet region
8.1 Horizontal air outlet
8.2 Treated air with attenuated and/or killed microorganisms, viruses and virions 8.2.1
8.2.1 Attenuated and/or killed microorganisms, viruses and virions
8.3 Horizontal disk-shaped cover of the horizontal air outlet 8.1
8.4 Horizontal air deflection disk
8.5 Horizontal surface of the disk-shaped cover 8.3
8.6 Air flowing out through the UVC-shielding, air-permeable grid arrangement 7.1
8.7 Circumferential horizontal separation point between the acoustophoresis region 6 and the tubular outlet region 7
9 Vertical outer wall of the drum-shaped device 1 according to the invention
B Stand space
E Electronics
F Wings of the horizontal axial rotor V
M Electric motor with speed control
V Axial rotor

### DETAILED DESCRIPTION OF THE FIGURES

### The Device 1 According to the Invention and According to FIGS. 1 and 2

The drum-shaped device 1 according to the invention had a vertical height of 1000 mm and a horizontal diameter of 200 mm. The wall thickness of the outer wall 9 made of anodized aluminum was 5 mm. Its outer surface was coated with a cream-colored top coat. The outer wall 9 was composed of the outer walls of the four symmetrically arranged, 20 mm high, circular-segment-shaped feet 2.4, between which the air contaminated with aerosols containing microorganisms, viruses and virions was sucked to the circular horizontal intake opening 2.3 with a circular circumference, the 40 mm high tubular intake region 2.1, the 100 mm high tubular air conveying region 3, the 370 mm high tubular UVC region 4, the 80 mm high tubular power supply region 5, the 300 mm high tubular acoustophoresis region 6, the 40 mm high tubular air outlet region 7, and the 30 mm high air outlet region 8. The circular-segment-shaped feet 2.4 were plug connections connected to the lower edge of the tubular wall of the intake region 2. The walls of the tubular regions 2; 3; 4; 5; 6; 7; 8 were connected at the separation points 3.1; 4.8; 5.3; 6.7; 7.2; 8.7 with flat bayonet connections. These bayonet connections could again be easily disconnected from each other by turning for maintenance and repair of the device 1 according to the invention.

The device 1 according to the invention had, at the necessary and appropriate places, a connection for the operating current and for charging a rechargeable battery, LED-function displays, controllers for the electronics E, for the radiation intensity of the UVC light source 4.1, the drive and speed control of the axial rotor M and the intensity of the standing acoustic ultrasonic fields in the acoustophoresis devices 6.6 as well as the necessary electrical lines. For the sake of clarity, these components have not been shown.

The contaminated air 2.2 was drawn through an intake opening 2.3 and through a multilayer, UVC-shielding, air-permeable grid arrangement 2.5 in the intake region 2, The grid arrangement 2.5 consisted of seven perforated sheets made of anodized aluminum, arranged in parallel on top of each other, with their air passages arranged in a staggered manner.

For conveying the sucked contaminated air 2.2 from the intake region 2 into the further regions of the device 1 according to the invention, the EC radial module—RadiCal® from ebm-papst Mulfingen GmbH & Co, KG was used as the axial rotor (V; M; F).

A Philips TUV PL-L 24W 4P 2G11 disinfector with two filaments 4.2 and the following characteristics was used as UVC light source 4.1:

Electrical Characteristics
Lamp wattage; 24 W
Voltage: 87 V

UV-Related Features
UV-C radiation: 7.1 Watt

Product Dimensions
Length base to base; 290 mm
Installation length: 315 mm
Total length C: 320 (max) mm
Diameter D: 39 (max) mm

Below the UVC light source 4, a first circumferential angled air guide ring 4.8 made of anodized aluminum sheet was arranged. It extended diagonally upwards and merged into a circumferential horizontal ring which ended 5 mm from the inner wall. Above this another circumferential, planar, horizontal aluminum ring of the inner wall of the UVC region 4 was attached, which ended 10 mm from the slope of the second angled air guide ring 4.10. The distance of the outer edges of the circumferential horizontal ring of the angled air guide ring 4.10 from the inner wall of the UVC region 4 was also 5 mm. This arrangement was attached to four symmetrically arranged vertical support rods 4.3 made of 3 mm diameter aluminum tubes. The support rods themselves were inserted into matching recesses in the support of the horizontally mounted axial rotor V. At their other ends they were attached to the ring-shaped holder 5.2 of the power supply 5.1 for the UVC light source 4.1.

Along the length of the emitter of the UVC light source 4.1, on the four parallel support rods 4.3 there were attached
(i) a planar aluminum ring 4.4 extending up to 2 mm to the outer side of the UVC light source 4.1 with in each case a circumferential air passage of a clear width of 5 mm between the outer edge and the inner wall of the UVC region 4 and
(ii) a planar horizontal aluminum ring 4.5 flush with the inner wall of the UVC region 4 and extending up to 2 mm to the outer side of the UVC light source 4.1.

Due to this arrangement, the path and thus the dwell time of the guided airflow 4.7 through the UVC region 4 was significantly prolonged, which is why the contaminated air 2.2 was exposed to UVC radiation for much longer than in the case of a laminar flow past the UVC light source 4.1 (cf. FIG. 2: 4.11; hv).

The air 6.5.1 irradiated with UVC radiation entered the two tubular acoustophoresis devices 6.6 arranged parallel to each other in the acoustophoresis region 6 through two inlet openings 6.5 with a circular circumference in the holder and power supply 5.1. The two acoustophoresis devices 6.6 had a length of 300 cm. The thickness of their closed walls 6.3 was 9 mm, and the inner diameter of the flow tube 6.6.2 was 72 mm. In each of the walls 6.3, eight arrangements of four ultrasonic emitter-receivers 6.1 arranged crosswise opposite each other were arranged one above the other at a distance of 20 mm, so that the two standing ultrasonic waves (6.2; 6.3) each had a common wave node 6.3. The ultrasonic emitter-receiver 6.1 was a columnar piezo-ultrasonic emitter of the type MCUST14A40S0RS of a diameter of 14 mm and a height of 9 mm, a central frequency of 40 kHz and a power level of 90 dB. They were glued with a polydimethylsiloxane adhesive into the corresponding openings in the walls 6.3. Their electrical connections faced outward and were connected to electronics E. All piezo ultrasonic transmitters 6.1 were glued into the closed walls 6.6.3 in such a way that they closed with their inner sides as planar as possible, so that no undesirable vortexes were formed in the dead volume area near the inner wall of the flow channels 6.6.4, The walls 6.6.3 were made of the sterilizable high-performance plastic polyethersulfone PES, which contained Hindered Amine Light Stabilizers HALS as UV light stabilizers.

In a second embodiment, wall-free flow regions 6.6.1 were used, with the piezo ultrasonic transmitters 6.1 arranged as described above, They were interconnected by insulated metal wires and holders. Since the aerosols, microorganisms, especially pathogenic microorganisms, viruses, virions, prions, allergens and pseudoallergens and/or their residues and/or decomposition products irradiated with UVC radiation migrated to the wave nodes 6.3 anyway, there was no difference in the mode of operation and effect of the two embodiments. An advantage of the second embodiment was that no UV light stabilizers had to be used.

In both embodiments of the acoustophoresis region 6, the aerosols, microorganisms, in particular pathogenic microorganisms, viruses, virions, prions, allergens and pseudoallergens and/or their residues and/or decomposition products irradiated with UVC radiation accumulated in and around the wave nodes 6.3 during operation due to the sound pressure and were ground there so to speak by the sound pressure, i.e., they aggregated or agglomerated, they were torn or pulverized and/or further decomposed, so that at most attenuated and/or killed and/or chemically and/or physicochemically modified microorganisms, in particular pathogenic microorganisms, viruses, virions, prions, allergens and pseudoallergens and/or their residues and/or decomposition products remained. These were discharged with the air through the air outlets 6.4, after which the outflowing air 8.6 was discharged into the room through the UVC-shielding, air-permeable grid arrangement 7.1, which had the same structure as the grid arrangement 2.5, through the horizontal air deflection disk 8.4 through the horizontal air outlet 8.1 bounded by the horizontal disk-shaped cover 8.3.

The air 8.2 discharged into the room contained the attenuated and/or killed and/or chemically and/or physicochemically modified microorganisms, in particular pathogenic microorganisms, viruses, virions, prions, allergens and pseudoallergens and/or their residues and/or decomposition products 8.2.1, from which there was no longer any risk of infection On the contrary, experiments with animals such as pigs, which are susceptible to infections, have shown that the resulting room air strengthened the animals' resistance to infection. Without being bound to a theory, it is suspected that the attenuated and/or killed and/or chemically and/or physicochemically modified microorganisms, in particular pathogenic microorganisms, viruses, virions, prions, allergens and pseudoallergens and/or their residues and/or decomposition products 8.2.1 apparently caused a strengthening of the immune system.

Preferably, the method according to the invention for attenuating and/or killing and/or chemically and/or physically chemically modifying microorganisms, in particular pathogenic microorganisms, viruses, virions, prions, allergens and pseudoallergens and/or their residues and/or decomposition products is carried out with the device 1 in the following manner:
(A) Suction of air 2.2 containing microorganisms, in particular pathogenic microorganisms, viruses, virions, prions, allergens and pseudoallergens and/or their residues and/or decomposition products and/or air 2.2 containing aerosols with microorganisms, in particular pathogenic microorganisms, viruses, virions, prions, allergens and pseudoallergens and/or their residues and/or decomposition products through the at least one intake opening 2.3 of an air-permeable intake region 2;
(B) conveying the air 2.2 through at least one air conveying region 3 with the aid of at least one axial rotor V with speed control or at least one controllable fan into at least one irradiation region 4;
(C) for attenuation and/or killing and/or chemically and/or physicochemically modifying the microorganisms contained in the air 2.2, in particular pathogenic microorganisms, viruses, virions, prions, allergens and pseudoallergens and/or their residues and/or decomposition products by irradiating the air 2.2 in the at least one irradiation region 4 with the actinic radiation of at least one UVC light source 4.1;
(D) entry of the resulting air 6.5.1 treated with actinic radiation through at least one opening 6.5 into at least one acoustophoresis region 6 with at least one acoustophoresis device 6.6;
(E) generating at least one controllable, stationary, acoustic ultrasonic field in the at least one acoustophoresis device 6.6 for acoustophoretic treatment of the air 6.5.1 flowing through;
(F) acoustophoretic treatment of the aerosols, attenuated and/or killed and/or chemically and/or physicochemically modified microorganisms, in particular pathogenic microorganisms, viruses, virions, prions, allergens and pseudoallergens and/or their residues and/or decomposition products 8.2.1. contained in the air 6.5.1. flowing through in the stationary acoustic ultrasonic field for the generation of acoustophoretically treated air 8.6;
(G) discharging the acoustophoretically treated air 8.6 from at least one air outlet 6.4 of the at least one acoustophoresis device 6.6 through at least one air outlet region 7 shielding actinic radiation, and
(H) discharging the air 8.2; 8.2.1 treated with actinic radiation and acoustophoretically from at least one air outlet region 8 directly into the environment or via at least one downstream device for filtration.

1. A device (1) shielded against the emission of actinic radiation for attenuating and/or killing and/or chemically and/or physicochemically modifying microorganisms, viruses, virions, prions, allergens and pseudoallergens and/or their residues and/or decomposition products and/or for blocking their transmission pathways, the device comprising
at least one intake region (2) that shields the actinic radiation and having at least one intake opening (2.3) to receive contaminated air (2.2),
at least one heatable and/or coolable air conveying region (3) which is detachably connected to the intake region (2) at a first circumferential separation point (3.1), and comprises at least one holder (3.2) for at least one axial rotor (V) driven by an electric motor (M) with speed control and having at least two rotor blades (F) or at least one controllable fan,
at least one irradiation region (4) having at least one ultraviolet-C (UVC) light source (4.1) that is a source of actinic radiation, wherein the at least one irradiation region (4) is detachably connected to the at least one air conveying region (3) at a second circumferential separation point (4.8),
at least one power supply region (5) having at least one circumferential planar holder (5.2) with power lines for a power supply (5.1) of the at least one UVC source (4.1) and having openings (6.5) for the entry of irradiated air (6.5.1) into at least one acoustophoresis region (6), wherein the at least one power supply region (5) is detachably connected to the at least one irradiation region (4) at a third circumferential separation point (5.3),
at least one acoustophoresis region (6) which is detachably connected to the power supply region (5) at a fourth circumferential separation point (6.7) and, comprising at least one transducer operable to generate at least one controllable, stationary, acoustic ultrasound field, at least one acoustophoresis device (6.6) having:
at least one wall-free flow region (6.6.1) and/or having at least one flow tube (6.6.2) with a closed wall (6.6.3), which encloses at least one flow channel (6.6.4), for the throughflow of the air (6.5.1) treated with actinic radiation, wherein
the at least one wall-free flow region (6.6.1) is enclosed by at least two pairs of mutually associated and mutually opposing ultrasonic emitters (6.1) or ultrasonic emitter-receivers (6.1) and/or by at least two pairs each of an ultrasonic emitter (6.1) or ultrasonic emitter-receiver (6.1) of ultrasonic waves (6.2; 6.3) and in each case a reflector (6.1) of ultrasonic waves (6.2; 6.3) associated therewith and opposite thereto and/or in which at least two ultrasonic emitters (6.1) or ultrasonic emitter-receivers (6.1) selected from the group consisting of standing, modulated and unmodulated longitudinal waves and transverse waves and their harmonics are arranged centrally in the at least one wall-free flow region (6.6.1), and wherein
the at least one flow tube (6.6.2) has a closed wall (6.6.3) which on its outer side and/or its inner side and/or in the respective closed wall (6.6.3) itself has at least two pairs of mutually associated and mutually opposite ultrasonic emitters (6.1) or ultrasonic emitter receivers (6.1) and/or at least two pairs of in each case one ultrasonic emitter (6.1) or ultrasonic emitter-receiver (6.1) and in each case one reflector (6.1) associated therewith and opposite thereto, wherein
the respective at least two pairs, as viewed in the direction of flow, are arranged one behind the other or are arranged in such a way that imaginary connecting lines between the respective at least two pairs cross at an angle of 90° and/or wherein
at least two ultrasonic emitters (6.1) or ultrasonic emitter-receivers (6.1) of ultrasonic waves (6.2; 6.3), selected from the group consisting of standing, modulated and unmodulated longitudinal waves and transverse waves and their harmonics, are arranged centrally in the at least one flow tube (6.6.2), and
comprising at least one air outlet (6.4) for discharging the acoustophoretically treated air (8.6),
at least one electronics (E) protected by a circumferential, planar, shielding (6.8) for generating, monitoring and stabilizing at least one feedback loop for adjusting and stabilizing the stationary acoustic ultrasonic field,
at least one air outlet region (7) that shields the actinic radiation, which is detachably connected to the at least one acoustophoresis region (6) at a fourth circumferential separation point (7.2), as well as
at least one air outlet region (8) for the treated air (8.2) containing the attenuated and/or killed and/or chemically and/or physicochemically modified microorganisms, viruses, virions, prions, allergens and pseudoallergens and/or their residues and/or decomposition products (8.2.1), which air outlet region (8) is detachably connected to the air outlet region (7) at a fifth circumferential separation point (8.7).

2. The device (1) according to claim 1, wherein the actinic radiation is blue light and/or UVC radiation.

3. The device (1) according to claim 1, wherein
(i) the device is arranged vertically, obliquely or horizontally in space and/or in that
(ii) the device has an outer wall (9) that has a circular, oval, elliptical, quadrangular, pentagonal, hexagonal or octagonal outline and/or in that
(iii) the device is constructed of materials that are stable and/or stabilized against actinic radiation.

4. The device (1) according to claim 1, wherein the at least one irradiation region (4) comprises at least three support rods (4.3) extending parallel to the at least one radiation source (4.1) for at least two pairs each of (i) planar metal rings (4.4), which are disposed one above the other in parallel, reach close to the outer side of the at least one radiation source (4.1) and each comprise a circumferential air passage between the outer edge and the inner wall of the irradiation region (4), and (ii) planar, horizontal metal rings (4.5) flush with the inner wall of the irradiation region (4) and extending as far as close to the outer side of the at least one radiation source (4.1), wherein the at least three parallel support rods (4.3) are anchored to or in the at least one holder (3.2).

5. The device (1) according to claim 1, wherein the at least one intake region (2) and the at least one air outlet region (7) each have at least one air-permeable UVC shield (2.5; 7.1).

6. The device (1) according to claim 5, wherein the air-permeable UVC shields (2.5; 7.1) are selected from the group consisting of at least two parallel superimposed grids, perforated plates, perforated screens and lamella arrangements made of metals, metal-coated plastics and window glass, the air passages of which are arranged in a staggered manner, macroporous carbon sponges, macroporous glass frits and plates with vertically aligned zigzag-shaped channels arranged parallel to one another.

7. The device (1) according to claim 1, wherein the ultrasonic waves (6.2; 6.3) have a frequency of 1 kHz to 800 MHz and the stationary acoustic ultrasonic field has an energy input of 0.25 W to 1 kW at a power level of 40 to 250 dB.

8. The device (1) according to claim 1, wherein the ultrasonic emitters (6.1) from the group consisting of loudspeakers, vibrating diaphragms, piezoelectric loudspeakers, sound transducers, virtual sound sources, immersion coils, magnetostatic loudspeakers, ribbon, foil and jet tweeters, horn drivers, bending wave transducers, plasma loudspeakers, electromagnetic loudspeakers, exciters, ultrasonic transducers and phantom sound sources.

9. The device (1) according to claim 1, wherein (i) the ultrasonic emitters (6.1) and the ultrasonic emitter-receivers (6.1) are sound-decoupled and vibration-decoupled from their holders and/or (ii) the reflectors (6.1) are selected from the group consisting of planar, concave and convex sound reflectors.

10. The device (1) according to claim 1, wherein at the separation points (2.1; 3.1; 4.8; 5.3; 6.7; 7.1; 8.7) the regions (2; 3; 4; 5; 6; 7; 8) are joined together by bayonet connections, screw connections, flange connections and/or plug connections.

11. The device (1) according to claim 1, wherein at least one device for filtration, selected from the group consisting of EPA, HEPA, ULPA, medium and activated carbon filters that are not coated and/or coated with biocides, is provided downstream of the at least one air outlet region (8).

12. A method for attenuating and/or killing and/or chemically and/or physically chemically modifying microorganisms, in particular pathogenic microorganisms, viruses, virions, prions, allergens and pseudoallergens and/or their residues and/or decomposition products and/or for blocking their transmission pathways, comprising:
(A) receiving air (2.2) containing microorganisms, in particular pathogenic microorganisms, viruses, virions, prions, allergens and pseudoallergens and/or their residues and/or decomposition products and/or air (2.2) containing aerosols with microorganisms, in particular pathogenic microorganisms, viruses, virions, prions, allergens and pseudoallergens and/or containing their residues and/or decomposition products through the at least one intake opening (2.3) of an air-permeable intake region (2) shielding the UVC radiation,
(B) conveying the air (2.2) through at least one heatable and/or coolable air conveying region (3) with the aid of at least one axial rotor (V) with speed control or at least one controllable fan (V) into at least one irradiation region (4),
(C) attenuating and/or killing and/or chemically and/or physicochemically modifying the microorganisms, viruses, virions, prions, allergens and pseudoallergens and/or their residues and/or decomposition products contained in the air 2.2 by irradiation of the air (2.2) in the at least one irradiation region 4 with actinic radiation from at least one source of actinic radiation (4.1),
(D) entering of the resulting air (6.5.1) treated with actinic radiation through at least one opening (6.5) into at least one acoustophoresis region (6) with at least one acoustophoresis device (6.6),
(E) generating at least one controllable, stationary, acoustic ultrasonic field in the at least one acoustophoresis device (6.6) for acoustophoretic treatment of the air (6.5.1) flowing through,
(F) acoustophoretically treating of the aerosols, attenuated and/or killed and/or chemically and/or physicochemically modified microorganisms, viruses, virions, prions, allergens and pseudoallergens and/or their residues and/or decomposition products (8.2.1) contained in the air (6.5.1) flowing through in the controllable stationary acoustic ultrasonic field for the generation of acoustophoretically treated air (8.6),
(G) discharging the acoustophoretically treated air (8.6) from at least one air outlet (6.4) of the at least one acoustophoresis device (6.6) through at least one air outlet region (7) shielding the actinic radiation, and
(H) discharging the air (8.2; 8.2.1) treated with actinic radiation and acoustophoretically from at least one air outlet region (8) directly into the environment or via at least one downstream device for filtration.

13. The method according to claim 14, wherein the sonic pressure in the method steps (E; F) is adjusted such that particles and/or fragments of microorganisms, viruses, virions, prions, allergens and pseudoallergens with a weight-average molecular weight of 5 kDa to 10 kDa are obtained.

14. The method according to claim 12, wherein at least one device (1) according to claim 1 is used.

15. The method according to claim 12 wherein the method is used for the treatment of the air in living rooms, sickrooms, operating theaters, treatment rooms in medical practices and physiotherapeutic facilities, laboratories of all kinds, inns, restaurants, bistros, hotel rooms, schoolrooms, classrooms, gyms, trains, cars, buses, cabs, caravans, mobile homes, camping tents, airplanes, ship cabins, offices, conference rooms, meeting rooms, theaters, cinemas, ship terminals, railroad stations, airport terminals, elevators, workshops, factory halls, staircases, stores and animal pens.

16. The device according to claim 1 wherein the device is used for the treatment of the air in living rooms, sickrooms, operating theaters, treatment rooms in medical practices and physiotherapeutic facilities, laboratories of all kinds, inns, restaurants, bistros, hotel rooms, schoolrooms, classrooms, gyms, trains, cars, buses, cabs, caravans, mobile homes, camping tents, airplanes, ship cabins, offices, conference rooms, meeting rooms, theaters, cinemas, ship terminals, railroad stations, airport terminals, elevators, workshops, factory halls, staircases, stores and animal pens.

17. The device according to claim 1 wherein, as seen in a direction of flow of the contaminated air (2.2)—first comprises the at least one acoustophoresis region (6) and then the at least one irradiation region (4), or first the at least one irradiation region (4), then the at least one acoustophoresis region (6) and finally at least one further irradiation region, or first the at least one irradiation region (4), then the at least one acoustophoresis region (6), then at least one further irradiation region (4) and finally at least one further acoustophoresis region (6), or first at least one acoustophoresis region (6), then at least one irradiation region (4), then at least one further acoustophoresis region (6) and finally at least one further irradiation region (4).
